(19) United States
(12) Patent Application Publication
(10) Pub. No.: US 2004/0205984 A1
(43) Pub. Date: Oct. 21, 2004

(54) Anti-roll arch support insole

(76) Inventor: John C. Hardt, Belton, TX (US)

Correspondence Address: Sidley Austin Brown & Wood, Suite 3400, 717 N. Harwood, Dallas, TX, 75201, US

(21) Appl. No.: 10/842,341

(22) Filed: May 10, 2004

Related U.S. Application Data
(63) Continuation of application No. 10/073,843, filed on Feb. 11, 2002, now abandoned.

(51) Int. Cl. A61F 5/14; A43B 7/14; A43B 7/16

(52) U.S. Cl. 36/140; 36/173; 36/178

(57) ABSTRACT

A shoe insole having an axis and an arch area with an upper surface and a bottom. The insole has three or more supporting ridges, positioned under the upper surface in the arch area, to provide support to a wearer's arch while allowing the insole to remain flexible as the wearer walks.

### CROSS REFERENCE TO RELATED APPLICATION

This application is a continuation of copending U.S. application Ser. No. 10/073,843, filed on Feb. 11, 2002, entitled ANTI-ROLL ARCH SUPPORT INSOLE, naming as inventor John C. Hardt.

### TECHNICAL FIELD

The present invention relates to an insole, in particular, an insole having an arch support and an anti-roll peripheral edge portion.

### BACKGROUND OF THE INVENTION

The present invention relates to shoe insoles, and more particularly, to improved insoles, particularly adapted for people with fallen arches. The insoles of the present invention provide comfort and support to individuals having fallen arches and also provide resistance to the tendency for the foot of such individuals to roll.

Previously, insoles of various designs have been utilized. Some insoles are simply flat sheets cut in the shape of the foot, and other insoles are thermoformed to the general shape of the foot. Additionally, it is known to place shock absorbing material in the heel area. Many insoles are designed to be loosely inserted into athletic and other shoes. Such insoles may be used as original equipment manufacture items to be placed in the shoe at the time it is manufactured, or as replacement or substitute insoles for those supplied with a pair of shoes.

In the past, insole design has frequently been tailored to various athletic or occupational requirements. Few insoles have been designed to specifically address the problems of individuals with fallen arches. Pes Planus or flatfoot (fallen arches) is the most common foot condition in patients of all ages. It is characterized by hindfoot valgus, forefoot abduction, and decrease in the height of the medial arch. In the past, rigid orthotics have been provided, but they have the disadvantage of hindering the natural movement of the foot. Such hard rigid supports limit performance and promote foot fatigue.

The present invention is addressed to the needs of indiviuals with fallen arches and provides several advantages. The present invention allows the insole to stay flexible and bend with the foot while continuously supporting the arch and not impeding the foot's movement. The arch design of the present invention minimizes the compression of the arch area and prevents the foot from rolling inward. The invention can also provide a small support column on the edge opposite the arch to counteract rolling to the outside. The preferred design provides a centering action to reduce ankle strain and foot roll. The present invention offers many advantages which can include firm support for the arch, resistance to the rolling of the foot in the shoe and shock absorption in the heel area.

### BRIEF DESCRIPTION OF THE DRAWINGS

The present invention will be better understood with reference to the accompanying drawings in conjunction with the detailed description. The drawings of the detailed descriptions are of preferred embodiments of the invention and thus, are not to be considered limiting.

FIG. 1 is a top view of one embodiment of an insole of the present invention;

FIG. 2 is a bottom view of FIG. 1;

FIG. 3 is a bottom view of another embodiment of the present invention being a three-quarter length insole;

FIG. 4a is a cross sectional view of FIG. 2 at line 4a-4a;

FIG. 4b is a cross-sectional view of FIG. 3 at line 4b-4b;

FIG. 5 is a side view of the embodiment of FIGS. 1 and 2;

FIG. 6 is a side view of another embodiment of the invention;

FIG. 7 is a partial side view of an arch of another embodiment of the present invention; and

FIG. 8 is a partial cross-sectional view along line 8-8 of FIG. 7.

FIG. 9 is a partial bottom view of arch area 24 illustrating the generally triangular shape of supporting ridge 28.

### SUMMARY OF THE INVENTION

The present invention is an insole which has a body defining the shape of an insole, having an axis and an arch area. The body has an upper surface for contact with the foot and a bottom. The insole has three or more supporting ridges positioned under the upper surface in the arch area. In a preferred embodiment, the insole includes a shock absorbing structure in the heel area. This shock absorbing structure may be in the form of a plurality of individual spaced-apart pods, or a section of shock absorbing material located in the heel area. Also in a preferred embodiment of the invention, the periphery of the insole extending along the edge opposite the arch and preferably the heel section, is scalloped and is substantially vertical.

### DETAILED DESCRIPTION

FIG. 1 is a top view of insole body 20. Like reference numbers and like drawings refer to corresponding elements. The insole defines an axis 22 which passes through the center of the heel and through a point between where the third or fourth toe would lie. The insole body 20 has an arch area 24 (shown in dashed lines) corresponding to the natural arch of a normal foot. Insole 20 has an upper surface 26 and has support ridges 28, (shown in phantom) underneath the upper surface in the arch area 24. Preferably, there are at least three supporting ridges 28. The supporting ridges preferably are positioned such that they are positioned at an angle 30, which is between about 800 to about 1000 from the axis. Most preferably, the supporting ridges are substantially perpendicular to the axis.

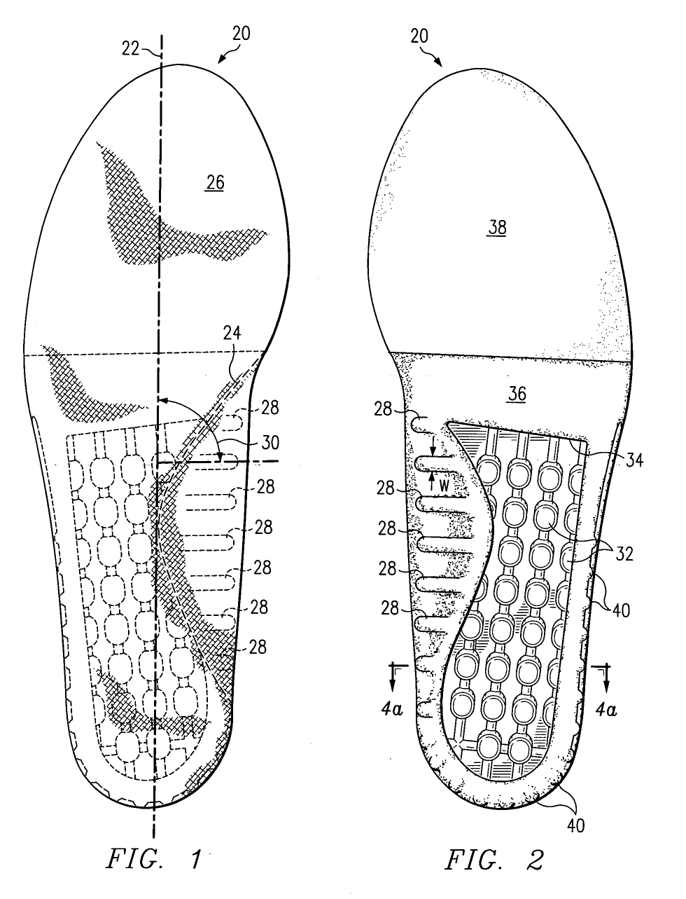

FIG. 2 is a bottom view of the insole of FIG. 1. FIG. 2 illustrates a plurality of shock absorbing pods 32 which are contained in a recessed area 34. Recessed area 34 can be located in the heel area or located in the heel and extend along the insole opposite the arch area 24. In the illustrated embodiment, the rear approximately three-quarters of the insole is a molded foam 36, and the front portion of the insole is formed by extending portion 38 of a neoprene sheet. In a preferred embodiment, the periphery around the heel has a substantially vertical edge defining a plurality of projections 40, forming a scalloped edge. This scalloped edge can extend along the side of the insole 20 opposite the arch area 24 as well.

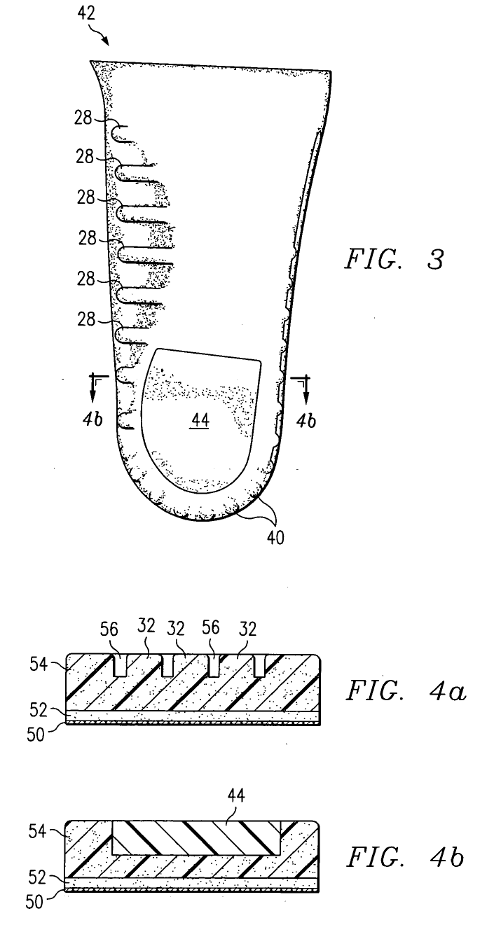

FIG. 3 is an alternate embodiment of the present invention. FIG. 3 illustrates insole body 42. Insole body 42 varies from insole body 20 in that insole body 42 is a three-quarter insole having the shape of the rear approximately three-quarters of the foot. This insole has an axis which, as in the other embodiment, passes through the center of the heel and through a point between where the third and fourth toe of a user would be during use if the three-quarter insole were extended to a full length insole. This embodiment has supporting ridges 28 and can also have projections 40. Insole body 42 illustrates an alternate design to provide shock absorption in the heel area. Insole 42 is provided with shock absorbing insert 44. Shock absorbing insert 44 may be of a sheet material, such as neoprene, around which the bottom of the insole is molded. Alternatively, shock absorbing insert 44 may be molded in place utilizing a suitable material as is known in the art.

FIG. 4a is a cross-sectional view from FIG. 2 along line 4a-4a and FIG. 4b is a cross-sectional view of FIG. 3 along line 4b-4b (it will be noted that both views are taken from a bottom view so that the top and bottom are reversed in the illustrations). Referring to FIGS. 4a and 4b, the cross section shows a top sheet 50. Top sheet 50 can be of any desired fabric. Generally, the function of the fabric top sheet 50 is to provide a wear surface and to increase aesthetic appeal of the insole. Any fabric known in the art may be used. Top sheet 50 is adhered, in the preferred embodiment, to the top layer 52 which is a sheet of neoprene. Adhered to the lower side of top layer 52 is molded bottom layer 54. Molded bottom section 54 is preferably polyurethane, but may be made of any moldable foam as is known in the art. Pods 32 are molded into the bottom. Pods 32 can be of any desired shape. They may be circular or other shapes. Spaces 56 in between the pods provide areas into which the pods can deflect and provide shock absorption. In a preferred embodiment, the pods are generally circular with a diameter of approximately 0.375 inches and a height of about 0.170 inches in the heel section. The spaces 56 between the pods can be from about 0.125 to 0.19 inches. The height of the pods 32 decreases as the thickness of the bottom molded layer 54 decreases as it approaches the front of the insole. In FIG. 4b, layers 50, 52 and 54 are present. In addition, bottom molded layer 54 contains shock absorbing insert 44. Shock absorbing insert can be a sheet material molded in place, or formed in situ by molding a material having the desired characteristics when the bottom layer is molded.

The insoles of the present invention may be made by known methods. Suitable methods are disclosed in U.S. Pat. Nos. 4,627,178, 4,674,204, 4,910,886, and 4,694,589.

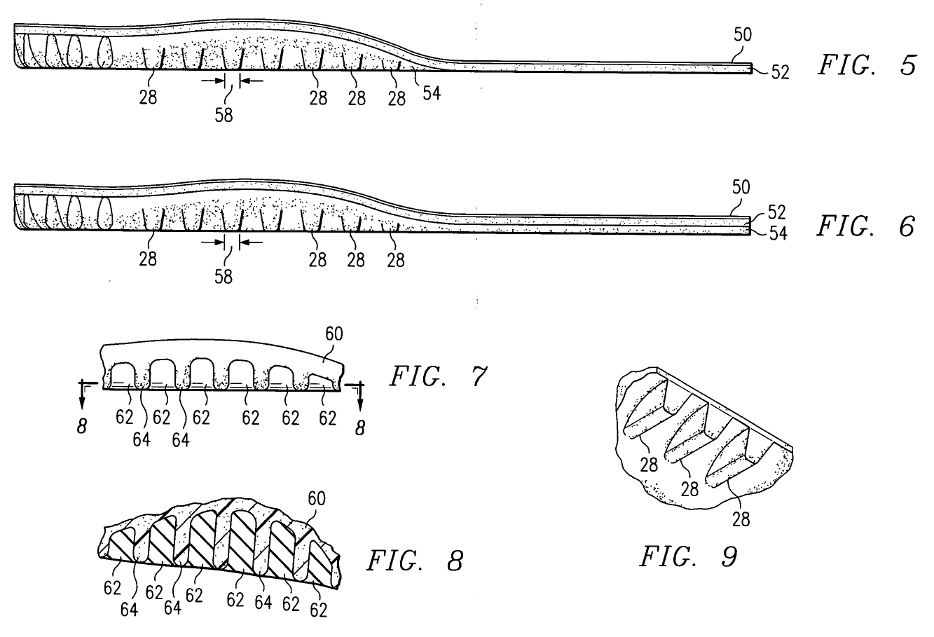

The insole body 20 has supporting ridges 28. The purpose of the supporting ridges 28 is to provide support to the arch of the wearer. When the bottom layer 54 is made of foamed polyurethane, the polyurethane develops a skin on the surface adjacent to the mold. This skin is much denser than the foamed polyurethane contained within the skin. It has been found that this skin helps maintain the supporting ridges 28 in their uncompressed dimension. Also, the skin and dimension of the ridges have been found to be important in minimizing or preventing deflection of the ridges when pressure is applied. It has been found that when the bottom layer 54 is molded polyurethane and the supporting ridges 28 are a molded integral part of the bottom layer 54, that the ridges should be from about 0.18 to about 0.35 inches wide (58 in FIGS. 5 and 6) when molded polyurethane is used. The space between the support ridges can be about 0.375 to about 0.500 inches. The number of ridges can vary with the size of the insole. In a preferred embodiment the ridges 28 are molded and form a portion of the bottom layer 54. The support ridges 28 should have a width which is sufficient to resist deflection of the ridge front to back as the wearer walks. The support ridges may be slightly compressible, so long as they maintain the arch shape and support the arch in the proper position. The support ridges will be of varying height and length depending upon their location in the arch area and the size of the insole. The height of each supporting ridge varies from the outside edge of the insole to the inside of the arch section. In the preferred embodiment, the ridges have a generally triangular or tapered shape with one side being the bottom, one side the outside edge, and the third side inside along what would be the bottom of the foot's arch. FIG. 9 illustrates the generally triangular shape of supporting ridge 28. For example, it has been found that for a man's size ten insole, made with a top layer of about 0.1875 inches neoprene and a molded bottom layer of polyurethane, that the largest ridge in the arch area has a height of about 0.43 inches on the outside edge of the insole, and a length of about 0.75 inches along the bottom.

FIGS. 5 and 6 show side views of embodiments of the invention. This width 58 of the support ridges is preferably about 0.18 to about 0.35 inches to provide adequate support. The height of the ridges is variable and preferably follows the shape of the arch. There should be at least three ridges. Preferably, there are 3 to about 7 support ridges.

FIG. 5 shows the preferred embodiment of insole body 20, having a top sheet 50 of fabric, top layer 52 of neoprene and a molded bottom layer 54 of polyurethane. FIG. 6 shows an alternate embodiment in which a molded urethane lower layer 54 extends the full length of the insole. It is not necessary to employ a neoprene top layer 52, nor is it required to have a top fabric sheet 50. Alternate embodiments of the invention would include an insole made entirely of molded urethane, and an insole of molded urethane with a fabric top sheet.

The support ridges 28 are utilized to minimize or prevent the arch area of the insole 20 from deflecting as weight is applied. These support ridges 28 form a bridge between the shoe's sole and the arch area of the foot. The support ridge design of the present invention allows the insole 20 to stay flexible and bend with the foot, while continuously supporting the arch and not impeding the foot's movement. Thus, the natural movement of the foot is not impeded. It was found that the use of the support ridges 28 in the arch area 24 make it desirable to increase the support on the opposing edge of the insoles. Thus, in the preferred embodiment of the present invention, the peripheral outside edge opposite the arch and the edge in the heel section were designed with a scalloped edge which is substantially vertical to provide an anti-roll feature. The outside perimeter edge opposite the arch and preferably extending around the perimeter of the heel area has the scalloped or corrugated shape with small projections 40. This design produces a corrugated edge that is much stronger than a straight or radius edge. The projections have substantially vertical walls. This is in contrast to insoles marketed today which have radiused outside edges which will roll inward or outward as weight is applied near the edge. The scalloped edge with the plurality of projections is preferred. This design facilitates the use of the insole as a replacement insole which can be inserted into shoes made by different manufacturers by providing an area that the projections can compress into. This permits the insole body 20 to fit in shoes made by different manufacturers and yet provide the anti-roll feature. For original equipment manufacturing, the edges of the insole can be custom shaped to fit tightly in the shoe so that the insole does not roll.

FIG. 7 is a partial side view of the arch area of yet another embodiment of the present invention. In FIG. 7, the insole has a molded bottom layer 60. Separate support ridges 62 are embedded in the bottom layer 60 between extensions 64 in bottom layer 60. FIG. 8 is a partial cross section along line 8-8 of FIG. 7. In this embodiment bottom layer 60 can be of a highly compressible material, and support ridges 62 can be of a fairly rigid material. Thus, as the user walks, ridges 62 provide support to the wearer's arch and extensions 64 of bottom layer 60 can compress to allow the insole to flex as the wearer walks. Support ridges 62 can be of a material placed in the mold and bottom layer 60 molded around them, or can be formed in situ by molding a material of desired characteristics. This embodiment is less desirable because of the increased cost of manufacture.

Other features of insole which can be desirable are to mold the bottom surface so that it has a rough surface similar to fine sandpaper. This helps to prevent slipping of the insoles from front to rear in the shoe.

While the present invention has been described in relation to its preferred embodiment, the description is not intended to be limiting of the invention but rather to describe certain preferred embodiments. Thus, modifications to the preferred embodiments will be appreciated by those skilled in the art without departing from the invention.

I claim:

1. An insole comprising:
a) a body defining the shape of an insole having a periphery, an upper surface for contact with the foot of a user and a bottom surface for contact with the interior of a shoe, each of said upper and bottom surfaces comprising a heel area, a toe area, and a middle area between said toe and said heel area, said middle area comprising an arch area and an area opposite said arch area, said insole having an insole axis passing through the center of the heel area and extending through a point in the toe area where a third or fourth toe of a user would contact said insole in said toe area during use, and
b) three or more supporting ridges positioned along the bottom surface of said insole in said arch area, said supporting ridges comprising a substantially straight vertical outer side, an inside and a substantially straight bottom defining a generally triangular shape having a width extending from about 0.18 to about 0.35 inches, said outer side having a height greater than said inside thereby defining a tapered surface from said outer side to said inside,
whereby said supporting ridges support the arch of a wearer's foot while allowing said insole to remain flexible and bend without impeding natural foot movement.

2. The insole of claim 1, wherein a portion of said periphery comprises a heel periphery defining said heel area and wherein said heel periphery comprises a substantially vertical edge.

3. The insole of claim 2, wherein said substantially vertical edge comprises a plurality of projections extending from said upper surface, defining a scalloped edge.

4. The insole of claim 2 or 3, further comprising additional projections extending from said heel periphery to a portion of said periphery of said insole in the area of said middle area opposite said arch area.

5. The insole of claim 1, wherein the bottom surface comprises a plurality of pods integrally formed in said heel area.

6. The insole of claim 4, wherein the bottom surface comprises a plurality of pods integrally formed in said heel area.

7. The insole of claim 1, wherein the bottom surface comprises a plurality of pods integrally formed in said heel area and extending to said middle area opposite said arch area of said bottom surface.

8. The insole of claim 4 wherein the bottom surface comprises a plurality of pods integrally formed in said heel area and extending to said middle area opposite said arch area of said bottom surface.

9. The insole of claim 1, 2 or 3, further comprising a shock-absorbing insert in said heel area.

10. The insole of claim 4, further comprising a shock-absorbing insert in said heel area.

11. The insole of claim 1, 2, 3, 5 or 7, wherein the rear three-fourths of said insole is molded foam and the front one-fourth of said insole is formed by extendent portion of a neoprene sheet.

12. The insole of claim 4, wherein the rear three-fourths of said insole is molded foam and the front one-fourth of said insole-is formed by extendent portion of a neoprene sheet.

13. The insole of claim 6, wherein the rear three-fourths of said insole is molded foam and the front one-fourth of said insole is formed by extendent portion of a neoprene sheet.

14. The insole of claim 8, wherein the rear three-fourths of said insole is molded foam and the front one-fourth of said insole is formed by extendent portion of a neoprene sheet.

15. The insole of claim 9, wherein the rear three-fourths of said insole is molded foam and the front one-fourth of said insole is formed by extendent portion of a neoprene sheet.

16. The insole of claim 10, wherein the rear three-fourths of said insole is molded foam and the front one-fourth of said insole is formed by extendent portion of a neoprene sheet.

17. The insole of claim 1, 2, 3, 5 or 7, wherein said insole is a three-quarter insole comprising molded foam.

18. The insole of claim 4, wherein said insole is a three-quarter insole comprising molded foam.

19. The insole of claim 6, wherein said insole is a three-quarter insole comprising molded foam.

20. The insole of claim 8, wherein said insole is a three-quarter insole comprising molded foam.

21. The insole of claim 9, wherein said insole is a three-quarter insole comprising molded foam.

22. The insole of claim 10, wherein said insole is a three-quarter insole comprising molded foam.

23. The insole of claim 1, wherein each of said supporting ridges have a ridge axis which is positioned at about 90 degrees from said insole axis.

24. The insole of claim 1 wherein said each of said supporting ridges have a ridge axis which is positioned at an angle of from 80 to 100 degrees from said insole axis.

25. An insole comprising:
a) a top layer in the shape of an insole defining an axis and having a upper surface and a lower surface;
b) a lower member attached to said lower surface of said top layer, having an arch area and having a bottom; and
c) three or more supporting ridges positioned along the bottom surface of said insole in said arch area, said supporting ridges comprising a substantially straight vertical outer side, an inside and a substantially straight bottom defining a generally triangular shape having a width extending from about 0.18 to about 0.35 inches, said outer side having a height greater than said inside defining a tapered surface from outside to inside.

26. The insole of claim 25, wherein said top layer is fabric.

27. The insole of claim 25, wherein said lower member is of molded polyurethane.

28. The insole of claim 25, wherein said arch area comprises supporting ridges comprising a rigid material and the space between said supporting ridges is filled with extensions of a highly compressible material, whereby said supporting ridges provide support to wearer's arch and said extensions compress to allow insole to flex with natural foot movement.

29. An insole of claim 25, 26, 27 or 28, wherein said supporting ridges have a ridge axis which is positioned at an angle of from 80 to 100 degrees from said axis

30. An insole comprising:
a) top sheet having the shape of an insole having a top and a bottom:
b) a top layer attached to said bottom of said top sheet;
c) a bottom layer attached to the lower surface of said top layer, having an arch area and having a bottom; and
d) three or more supporting ridges positioned under said top sheet in said arch area, said supporting ridges comprising a substantially straight vertical outer side, an inside and a substantially straight bottom defining a generally triangular shape having a width extending from about 0.18 to about 0.35 inches, said outer side having a height greater than said inside defining a tapered surface from outside to inside.

31. An insole of claim 30, wherein said top layer is of neoprene.

32. An insole of claim 30, wherein said bottom layer is of molded polyurethane.

33. An insole of claim 31, wherein said bottom layer is of molded polyurethane.

34. An insole of claim 30, 31, 32 or 33, wherein said support ridges have a ridge axis which is positioned at an angle of from 80 to 100 degrees from said axis
